(19) United States
(12) Patent Application Publication
(10) Pub. No.: US 2009/0062783 A1
(43) Pub. Date: Mar. 5, 2009

(54) ICE TIP HYPODERMIC NEEDLE

(76) Inventor: William Y. Sun, Alexandria, VA (US)

Correspondence Address: CLYDE I. COUGHENOUR, 16607 SUTTON PLACE, WOODBRIDGE, VA, 22191, US

(21) Appl. No.: 11/846,166

(22) Filed: Aug. 28, 2007

(51) Int. Cl. A61B 18/02; A61F 7/12

(52) U.S. Cl. 606/24; 604/113

(57) ABSTRACT

An ice tip is placed on the front end of a hypodermic needle to cool the flesh during insertion. The ice tip front end is sharp, for penetration, and the rear is provided with a recess, for reception of a hypodermic needle front end. The mold for forming the ice tip can be cooled by a cryogenic fluid being conducted into a main mold surrounding a tip forming heat conducting mold that is used to form the ice tip pointed front end and the recessed rear end.

### BACKGROUND OF THE INVENTION

1. Field of the Invention

The invention is to a frozen water hypodermic needle tip to make an injection reasonably pain free.

2. Description of Related Art

The use of cold surfaces and cryogenic fluids to cool the skin where injection or cutting is to take place per se is old and obvious. Examples of prior art patents that use various means to cool the skin or needle include: U.S. Pat. No. 2,746,264 issued May 22, 1956, to F. G. Keyes; U.S. Pat. No. 3,333,587 issued Aug. 1, 1967 to R. D. Johnston; U.S. Pat. No. 3,351,063 issued Nov. 7, 1967, to Malaker et al; U.S. Pat. No. 3,399,675 issued Sep. 3, 1968, to C. W. Hill, U.S. Pat. No. 3,483,869 issued Dec. 16, 1969, to M. J. Hayhurst; U.S. Pat. No. 3,605,742 issued Sep. 20, 1971, to R. C. Tibbs; U.S. Pat. No. 4,646,735 issued Mar. 3, 1987, to J. S. Seney; U.S. Pat. No. 4,725,265 issued Feb. 16, 1988, to M. Sairenji; U.S. Pat. No. 5,236,419 issued Aug. 17, 1993, to J. S. Seney; U.S. Pat. No. 6,936,028 issued Aug. 30, 2005, to Hommann et al; U.S. Pat. No. 7,097,641 issued Aug. 29, 2006, to Arless et al.

### SUMMARY OF THE INVENTION

A hypodermic needle is provided with an ice tip for cooling and penetrating the skin of a patient. The tip is secured to the front end of a blunt or standard cannula. The ice tip is held on the cannula by a flange at the rear of the tip. The tip can be frozen at the place of manufacture or use, using a cryogenic fluid. On exposing the tip to the warmth of the atmosphere or body of a patient, the flange and tip begin to melt permitting the injection of a fluid through the cannula of a hypodermic needle.

### BRIEF DESCRIPTION OF THE DRAWINGS

FIG. 1 is a side cross-section of a modified hypodermic needle having an ice tip.

FIG. 2 is a side view and an ice tip separated from the hypodermic needle.

FIG. 3 is a side cross-section of a hypodermic needle tip mold for use with a cryogenic fluid.

FIG. 4 is a side cross-section of a standard hypodermic needle used with an ice tip.

### DESCRIPTION OF THE PREFERRED EMBODIMENTS

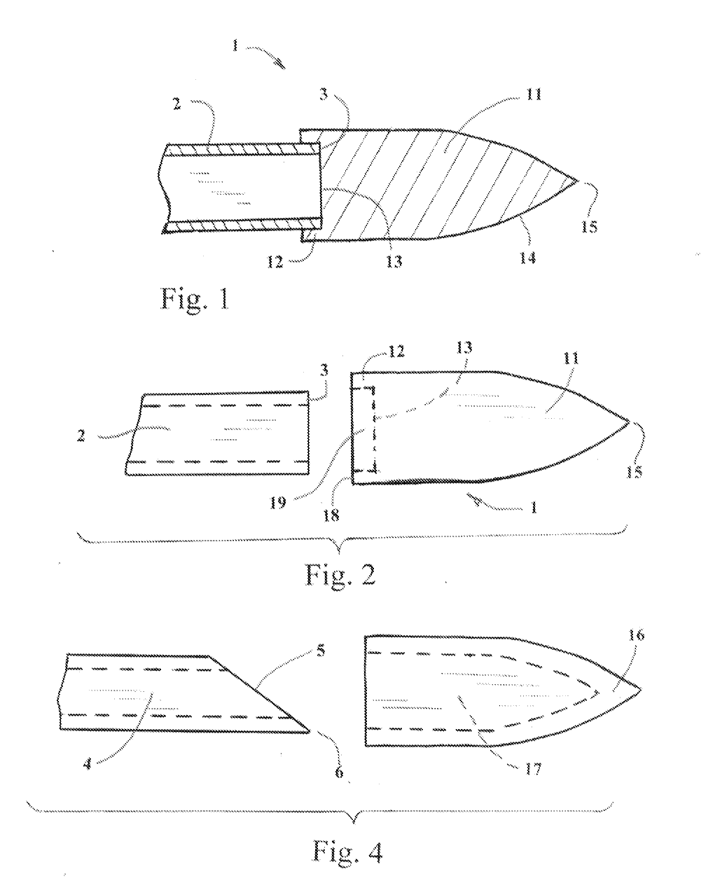

The invention is to a frozen hypodermic needle tip to make an injection as painless as possible. The needle tip is designed to be used with a modified needle structure or a standard needle structure. The ice tipped cannula 1 is shown in FIG. 1. A modified cannula shank 2 has a blunt front end 3 with an ice tip 11 secured to the front blunt end. The ice tip 11 has a rear flange 12 that fits over the front end 3 of the cannula 2. The flange has a thickness of from 0.010 to 0.025 inch. The front end of the cannula extends into the recess formed by the flange 12 to the recess base 13 in the ice tip. The recess has a depth of from 0.060 to 0.250 inches. The ice tip extends from the rear flange 12 rear extreme 18 to the forward front tip end 15 in straight or curved side surfaces 14. The ice tip has an overall length of from 0.250 to 1.250 inches.

It is known that a person suffers less or is not as sensitive to hypodermic needles where the skin or flesh surface is cold. The ice needle tip serves to cool the skin to reduce both pain and apprehension. Since the atmospheric temperature and body temperatures are both higher than the ice, as soon as the hypodermic ice tip is exposed it begins to melt the surface, The wet needle ice tip performs the function of assisting heat transfer from the flesh surface to the ice tip; and acting as a lubricant. The ice tipped cannula 1 is inserted using the tip front end 15. The rear flange of the ice tip is thin and quickly melts when inserted into the body or body part. With the flange melted, fluid can be injected through the cannula shank 2 past the blunt front end 3 and into the body or body part. With the ice tip body 11 having more mass, it takes longer to melt but, due to the relative overall size of the ice tip, there will be no adverse effect to the body part or blood therein

The ice tipped cannula 1 can be “factory manufactured” with the cannula shank 2 and ice tip body 11 formed together for both shipment and use or they can be placed together, for example, at the point of use.

In FIG. 2, a side view of the cannula shank 2 and ice tip 11 are shown separated as would be done if the ice tip were to be inserted at the point of use. The cannula shank front end 3 is pressed into the ice tip recess 19 to the base 13.

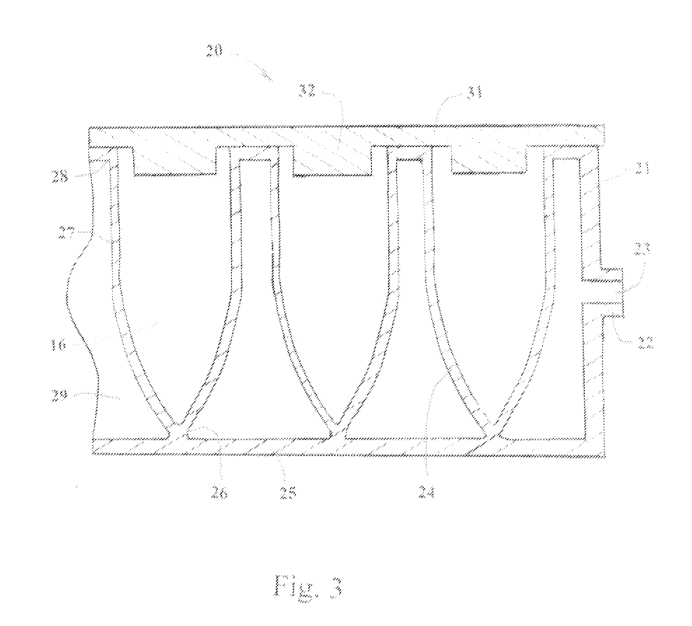

In FIG. 3, a cross-section of one example of a mold that could be used to manufacture the ice tip at a factory or point of use is shown. The primary mold 20 includes a cover 31 for the tip cavity 16 with projections 32, that can be tapered for easy removal, used to form a recess 19 in the tip rear base 13. The mold main body has ends 21 and bottom 25 and optional top 28. The end of the main body is provided with an access 22 having an access opening 23 for introduction of a cryogenic fluid with an exit opening provided (not shown). The individual tip molds 27 have tip shape openings 16 filled with water to be frozen. A point shape cavity 26 front end 24 extends to the main mold body bottom 25. The individual tip molds 27 are made of a highly thermally conductive material that may be the same as or different from that of the main mold body 20. The sides can be tapered for easy removal of the tip 16. The water in the tip shape opening 16 is frozen by introduction of a cold or cryogenic fluid into the mold through the opening 23 in the end 21 of the main mold body.

To avoid the need for refrigeration, a die can be provided with a cryogenic fluid dispenser for freezing water in the die 20 as shown in FIG. 3. To accommodate this, the die is provided with internal passages 28 for conducting cryogenic fluid past the cannula cavity 2 in the die to an exit opening 29 in the front end of the die. A cryogenic fluid adapter 30 closes the cannula cavity rear end. The cryogenic fluid adapter is provided with a threaded cryogenic fitting for supplying a cryogenic fluid from a cryogenic dispenser. An inlet 32 connects with a groove 33 extending around the base of the cryogenic adapter 30 that alines with the cryogenic passages 28 that extend around and along the cannula cavity 22 for removing heat from the cavity and any water or ice in the cannula cavity. The cryogenic fluid adapter 30 is secured on the die with adapter locks 34 that are locked and removed by turning the adapter 30 relative to the die 20. The die 20 can be slid off 38 the frozen hypodermic needle.

In FIG. 4 an alternate ice tip 16 is shown. The tip 16 is provided with a recess 17 that will accept a standard cannula shank 4 with a tapered front bevel 5 with point 6. The recess 17 extends into the ice tip from the rear surface for from 25% to 75% of the length of the ice tip.

It is believed that the construction, operation and advantages of this invention will be apparent to those skilled in the art. It is to be understood that the present disclosure is illustrative only and that changes, variations, substitutions, modifications and equivalents will be readily apparent to one skilled in the art and that such may be made without departing from the spirit of the invention as defined by the following claims.

1. An ice hypodermic needle tip comprising:
an elongated ice tip essentially the diameter of a hypodermic needle having a front end and a rear end;
said ice tip front end having a flesh penetrating point;
said ice tip rear end having a hypodermic cannula receiving recess.

2. An ice hypodermic needle tip as in claim 1 wherein:
said hypodermic cannula receiving recess is formed by a flange extending rearwardly from said ice base.

3. An ice hypodermic needle tip as in claim 2 wherein:
said flange extending rearwardly from said ice tip rear base has a thickness of from 0.010 to −0.025 inch.

4. An ice hypodermic needle tip as in claim 3 wherein:
said hypodermic cannula receiving recess has a depth of from 0.060 to 0.250 inches.

5. An ice hypodermic needle tip as in claim 4 wherein:
said hypodermic cannula receiving recess has a flat base extending perpendicular to the length of said elongated tip.

6. An ice hypodermic needle tip as in claim 5 wherein:
said elongated ice tip has an overall length of from 0.250 to 1.250 inches.

7. An ice hypodermic needle tip as in claim 3 wherein:
said hypodermic cannula receiving recess extends into said elongated ice tip centrally a distance of from 25% to 75% the length of said elongated tip for reception of a cannula shank tapered penetration tip.

8. An ice hypodermic needle tip as in claim 7 wherein:
said elongated ice tip has an overall length of from 0.0250 to 1.250 inches.

9. An ice hypodermic needle tip as in claim 1 wherein:
a cannula extends into said elongated ice tip recess.

10. An ice hypodermic needle tip as in claim 9 wherein:
said cannula front end is blunt with said blunt front end positioned against said recessed base of said ice tip.

11. An ice hypodermic needle tip as in claim 9 wherein:
said cannula front end is tapered to a point;
said cannula front end taper and point extend into said elongated ice tip recess.

12. An ice hypodermic needle tip as in claim 1 wherein:
said elongated ice tip is within a heat conducting tip mold.

13. An ice hypodermic needle tip as in claim 12 wherein:
said heat conducting tip mold forms said flesh penetrating tip front end and said rear hypodermic receiving recess.

14. An ice hypodermic needle tip as in claim 13 wherein:
said heat conducting tip mold is surrounded by a space within a primary mold.

15. An ice hypodermic needle tip as in claim 14 including;
an entrance into said primary mold for introduction of a cryogenic fluid into said space within said primary mold and around said heat conducting tip mold.

16. A hypodermic needle tip comprising:
an elongated tip essentially the diameter of a hypodermic needle having a front end and a rear end in the form of water within a heat-conducting tip mold;
the water front end is in the shape of a flesh penetrating point;
the water rear end is in the shape of a hypodermic cannula receiving rear end;
a cryogenic fluid means for contacting said heat conducting tip mold.

17. A hypodermic needle tip as in claim 16 including:
said heat-conducting tip mold having a flesh penetrating point front end shaped cavity and a rear hypodermic cannula receiving rear open end shape cavity;
a primary mold surrounding said heat-conducting tip mold and spaced from said heat conducting tip mold;
said cryogenic fluid means includes a cryogenic fluid passage within said primary mold that extends around said heat-conducting tip mold.

18. A hypodermic needle tip as in claim 17 wherein:
said primary mold cryogenic fluid passage has an access opening for introduction of a cryogenic fluid from outside said primary mold into said primary mold and around said heat-conducting tip mold.
